(19) United States
(12) Patent Application Publication
(10) Pub. No.: US 2021/0040429 A1
(43) Pub. Date: Feb. 11, 2021

(54) HOLDERS FOR BIOREACTOR SENSORS, BIOREACTORS HAVING SUCH HOLDERS, AND METHODS CULTURING BIOLOGICAL MATERIAL

(71) Applicant: SCHOTT AG, Mainz (DE)

(72) Inventors: Christian Ott, Ampfing (DE); Robert Hettler, Kumhausen (DE)

(73) Assignee: SCHOTT AG, Mainz (DE)

(21) Appl. No.: 16/989,701

(22) Filed: Aug. 10, 2020

(30) Foreign Application Priority Data
Aug. 9, 2019 (DE) 10 2019 121 541.2

(51) Int. Cl. C12M 3/00; C12M 1/26; C12M 1/36

(52) U.S. Cl. CPC C12M 23/48; C12M 41/48; C12M 33/00

(57) ABSTRACT

A bioreactor is provided that includes a container for receiving fluid media that contain biological material and a passage with a passage opening between an interior of the container and an exterior of the container. The bioreactor includes a holder configured to hold a sensor that extends at least partially in the passage opening of the passage. The holder has a sensor chamber and a fine-pored glass layer with a pore size of up to 0.2 μm. The fine-pored glass layer bounds the sensor chamber at least partially, whereby, in particular, an exchange of fluids between the interior of the container for receiving fluid media that contain biological material and the interior of the sensor chamber is made possible.

### CROSS REFERENCE TO RELATED APPLICATIONS

This application claims benefit under 35 USC 119 of German application 10 2019 121 541.2 filed on Aug. 9, 2019, the contents of which are incorporated by reference.

### BACKGROUND

### 1. Field of the Invention

The invention relates to a holder for the sterile holding of a sensor for a bioreactor as well as a bioreactor having a holder for the sterile holding of a sensor and a method for propagating or culturing biological material. The holder is also referred to below as a holder, a sterile port, or a sterile sensor port and, in the scope of the present disclosure, a port is understood to mean a passage, in particular also a passage for fluid media. Methods for producing biological material, such as, for example, biotechnological production processes, which are also referred to as biotech production processes and which comprise the culturing of microorganisms and of animal and plant cells, are of increasing importance. These biotechnological production processes comprise, for example, the culturing of microorganisms and of animal and plant cells.

### 2. Description of Related Art

Conventionally, measuring probes are installed prior to the sterilization of a bioreactor, both in the case of disposable bioreactors, which are also referred to as single-use reactors, and in the case of bioreactors intended for multiple use, which, in standard practice, are also referred to as multi-use reactors.

Crucial for the development and control of biotech production processes are sterile conditions and real-time measurements of key parameters. For the production of biopharmaceuticals, in particular, there is a push for improvements in product yield and thus an increase in profit.

Without sterilization, culturing would be contaminated. The loss in yield would necessarily be the consequence of infection.

The yield of biotechnological methods often depends crucially on the determination of substrate and product concentrations. For this reason, samples are taken during the culturing. Here, the time expenditure is considerable, however. This is due to the sampling, which is routinely also associated with a risk of contamination, but is also due to resource-intensive off-line analysis.

In spite of these efforts, process control remains impaired by the lack of real-time data.

DE 10 2010 063 031 A1 shows a potentiometric sensor as well as a method for starting up operation of a potentiometric sensor. In order to make the utilization of a container that serves as a single-use fermenter or as a single-use bioreactor especially simple, the potentiometric probe disclosed in this patent can already be installed firmly in a wall of the container via a connection prior to sterilization by gamma radiation, for example, and can remain therein for the duration of storage and use.

Described in DE 10 2006 022 307 A1 is a single-use bioreactor with a reversibly, externally attachable sensor arrangement for measuring a physical variable of a medium contained in the bioreactor, wherein, in at least one peripheral line of the bioreactor that serves for the inflow and/or the outflow of a medium, a sensor adapter is integrated for mounting an electronic sensor arrangement that interacts with the medium that flows through the peripheral line via an inner interface of the sensor adapter. Because, in each case, the measurement can be made only in the peripheral line of the bioreactor, a process control inside the bioreactor is not possible with use of this arrangement.

DE 10 2010 037 923 A1 discloses a bioreactor arrangement for cells, which contains a sealed bioreactor, a cell pellet carrier for holding a cell pellet, and means for feeding nutrient solution to the cell pellet. This arrangement allows the contact-free measurement of the oxygen content. A laser is thereby used to excite oxygen probes to undergo phosphorescence. The emitted phosphorescence signal is received by the detector and transmitted to an electronic analysis unit. For this purpose, the bioreactor has translucent windows and the laser and the detector are arranged outside the bioreactor. An exchange of sensors or measuring devices is not described in this document.

DE 10 2011 101 108 A1 describes a transflection probe for performing a transflection measurement on a fluid that is present in a rigid container by use of a probe shaft that is furnished with a light guide path in its interior and at the front side of which is arranged an open flow chamber having a reflection plate lying opposite to the front side of the probe shaft. The probe shaft is designed as a rigid hollow body that is closed at its front side by a transparent window and, at its rearward end, has a first coupling device for the rigid coupling of a sensor module to the probe shaft. However, by this coupling device, the sensor module is joined in a fixed spatial constellation to the open flow chamber and, in particular, to the reflection plate lying opposite to the front side of the probe shaft, so that the changing of a sensor module at a probe or the alternating coupling of a sensor module at different probes of the same or different containers is made possible. A changing of sensors for measuring physical, chemical, or biological measurement variables is not disclosed.

Conventional sterile filters for cell suspensions are composed primarily of polyether sulfone, polypropylene, nylon, and nitrocellulose. They tend to clog owing to membrane fouling. As a result, these conventional sterile filters usually cannot be used for the required culturing time.

According to the prior art, an in-situ process control using non-sterilizable measuring probes is not available at the present time.

### SUMMARY

The invention is based on the object of providing a holder, also referred to as a sensor port, for the sterile holding of a sensor as well as a bioreactor that is provided therewith, by means of which, in each case, an in-situ process control using non-sterilizable measuring probes is made possible, without thereby impairing the sterile conditions inside the bioreactor, that is, without contaminating the bioreactor.

This object is achieved by the present disclosure.

A bioreactor according to the invention comprises a container for receiving fluid media that contain biological material as well as a passage with a passage opening between the interior of the container for receiving fluid media that contain biological material and the exterior of the container for receiving fluid media that contain biological material, wherein a holder for the sterile holding of at least one sensor extends at least partially in the passage opening of the passage, wherein the holder for the sterile holding of at least one sensor comprises a sensor chamber and a fine-pored glass layer with a pore size of up to 0.2 μm, wherein the fine-pored glass layer with a pore size of up to 0.2 μm bounds the sensor chamber at least partially, as a result of which, in particular, an exchange of fluids between the interior of the container for receiving fluid media that contain biological material and the interior of the sensor chamber is made possible.

The fine-pored glass layer forms, as first layer, a sterile boundary and, with pores of a pore size of up to 0.2 μm, can reliably prevent the bioreactor from becoming contaminated. This means that no microbes or contaminating substances can enter the bioreactor through these pores.

This layer is advantageously protected against membrane fouling by another, second layer made of porous glass.

Especially advantageous is the utilization of systems, in particular sensors, that are not sterilizable and cannot be calibrated in situ, but that make possible an increase in the efficiency and yield associated therewith.

These systems and sensors include biosensors, which, as a rule, are analyte-specific. For example, in the case of an unexpected behavior of eukaryotic cells during culturing, it is possible to exploit a preferred property of a sensor for the process control.

Biosensors themselves are not sterilizable and the use thereof therefore necessitates a sterile interface between the bioreactor and the sensor in order to maintain the sterility boundary.

The sterility challenge is also described, for example, in “Sterilization of enzyme glucose sensors: problems and concepts,” Th. von Woedtke, W.-D. Jülich, V. Hartmann, M. Stieber, P.U. Abel, Biosensors & Bioelectronics 17 (2002) 373-382.

In “Integrated multi-sensor system for parallel in-situ monitoring of cell nutrients, metabolites, cell density and pH in biotechnological processes,” Stefan Mrossa, Tom Zimmermann^{a}, Nadine Winkin^{b}, Michael Kraft^{c}, Holger Vogt^{a}, Sensors and Actuators B 236 (2016) 937-946, the necessity of a practicable in-situ measuring system is described, because, for complex products, such as, for example, antibodies, it is necessary to keep the culturing conditions as close as possible to an at least local optimum. Deviations from the optimum impair, for example, the glycosylation, with a loss of yield and quality associated therewith. Multi-sensor platforms make it possible to counteract these deviations in real time. The function and space requirement of a multi-sensor platform harmonize with the devices disclosed here, in particular the herein described holder for the sterile holding of at least one sensor, the holder also referred to as a sterile port, and make it possible through in-situ monitoring to increase efficiency, yield, and quality.

Preferably, the passage comprises a standard port, such as, for example, an Ingold port, a Broadly James port, a B. Braun safety port, or another corresponding standard port. These ports each have an opening of defined diameter, which, in a typical way, connects or opens up the interior of a bioreactor to its exterior.

In general, the container for receiving fluid media that contain biological material can be the container of a multi-use bioreactor.

Advantageously, the container for receiving fluid media that contain biological material comprises stainless steel or it is composed of stainless steel.

Alternatively, the container for receiving fluid media that contain biological material can also be the container of a single-use bioreactor.

In this case, it is advantageous when the container for receiving fluid media that contain biological material comprises a plastic, in particular a sterilizable plastic, or is composed of a plastic, in particular a sterilizable plastic. This plastic can comprise a polymeric material and, in particular, can be composed of a suitable material that withstands a gamma sterilization or a chemical sterilization with ETO.

Suitable as plastics are, for example, polyester elastomers containing EVOH (ethylene vinyl alcohol copolymer) or polyethylene. In this case, it is possible to use a mixture of materials in which, for example, a layer system has an outer layer that provides mechanical stability. A gastight intermediate layer can then follow an inner-lying biocompatible layer.

In order to the meet the stringent requirements for the production of biopharmaceuticals, the material that the bioreactor and the sensor holder each comprise can be chosen such that, in each case, it conforms to the following standards: FDA approved materials (ICH Q7A, CFR 211.65(a)—Code of Federal Regulations, USP Class, animal derivative free, bisphenol A free); EMA (European Medicines Agency) EU GMP Guide Part II approved materials; Sectoral chemical resistance—ASTM D 543-06; and Biocompatibility, e.g. referred to US Pharmacopeia or tests referred to ISO 10993.

In order to achieve favorable dimensional relationships and a mechanically stable mounting of the holder for the sterile holding of a sensor, which by abbreviation is also referred to as a sensor holder, and, in particular, of a sensor device held in the sensor holder, the holder for the sterile holding of at least one sensor or at least one sensor holder extends inside the passage in a form-fitting manner in relation to the passage.

It is especially advantageous when the bioreactor, together with the holder for the sterile holding of at least one sensor, can be autoclaved and, in particular, can be autoclaved while it is held at least partially in the passage opening of the passage. In this way, it is possible to rule out with high certainty any contamination with biologically active or biologically interacting material.

An advantageous embodiment comprises a sensor chamber and a fine-pored glass layer with a pore size of up to 0.2 μm, which at least partially bounds the sensor chamber, as a result of which, in particular, an exchange of fluids between the interior of the sensor chamber and the exterior of the sensor chamber is made possible.

It is also advantageous in this case when the exchange between the interior of the container for receiving fluid media that contain biological material and the interior of the sensor chamber is made possible, in particular when the holder for the sterile holding of at least one sensor is arranged in a passage opening of a passage of a bioreactor, as is disclosed in the present case.

When the fine-pored glass layer with a pore size of up to 0.2 μm forms a first layer, which is surrounded by a second porous layer, which has a pore size of 200-700 μm for a porosity of up to 85%, it is not only possible to reliably prevent any contamination of the bioreactor, but also, owing to the large surface of the second layer, to extensively suppress a fouling or clogging of the pores of the first layer.

The porosity ϕ of a layer, in particular a layer composed of glass, is regarded as meaning the ratio of the volume Vp of the pores present in the glass relative to the volume Vo of the glass without the pores and the following thus applies: ϕ=Vp/(Vo+Vp). Insofar as this value is given in percent, this statement then relates to the percent volume of the pores relative to the total volume of the glass.

In a preferred embodiment, the sensor chamber is an essentially cylindrical shape and the first layer and the second layer extend in the longitudinal direction of the cylindrical sensor chamber and, in particular, extend so as to terminate distally at the front side of the sensor chamber.

In this preferred embodiment, the first layer can be composed of a first glass or can comprise a first glass, and the second layer can be composed of a second glass or can comprise a second glass.

Alternatively, the first layer can also be composed of a first glass or can comprise a first glass, and the second layer can also be composed of the first glass or can comprise the first glass.

For an exchange of fluids that is as effective as possible and a sensor-based recording that is made in a manner that is as timely as possible, it is advantageous when the first layer and the second layer extend in the longitudinal direction continuously along the sensor chamber.

In another preferred embodiment, the first layer and the second layer do not extend continuously in the longitudinal direction, defining the sensor chamber, but rather the first layer and the second layer are held on an essentially tubular support and, in particular, are held on an essentially tubular support that comprises glass or is composed of glass.

By use of a piston that can move in the longitudinal direction, in particular with a piston carrier or a piston rod by means of which the piston can be moved in the longitudinal direction, it is possible to seal the sensor chamber against the outside of the container in a fluidtight manner and advantageously to position there, if need be, a sensor that is held at this piston.

For this purpose, the piston can advantageously have a sensor holder.

It is especially advantageous when the piston, together with the sensor holder, can be moved between a first position, which is associated with a sterilization or autoclaving, and a second position, which is associated with the sensor-based recording. In this way, the thermal load caused by the sterilization or autoclaving can be minimized or the sensor can even be removed during the sterilization or autoclaving of the sensor chamber.

When the piston has a place holder for at least one sensor, this place holder can then occupy the position of an otherwise used sensor during the autoclaving or the sterilization, for example.

In general, it is advantageous when the piston has a fastening for a sensor, in particular a standardized fastening for a sensor, since it is then possible, for example, to carry out a quick changing of sensors, in particular even sensors of different kinds.

Advantageously, the piston can comprise a plastic, in particular an ethylene propylene diene monomer (EPDM) rubber.

Alternatively, the piston can also comprise a ground glass piston, preferably with an 0-ring arranged on it.

For all embodiments of the bioreactor described above as well as for all embodiments of the holder described above, it can be particularly provided that the fine-pored glass layer with a pore size of up to 0.2 μm has a coefficient of permeability (also referred to as a hydraulic conductivity) that, for water, is at least 5*10⁻⁶ m/s, preferably at least 2*10⁻⁵ m/s. In particular, it can be provided that the coefficient of permeability for water lies in the range of 5*10⁻⁸ m/s to 2*10⁻⁴ m/s and preferably lies in the range of 2*10⁻⁶ m/s to 2*10⁻⁴ m/s.

For one exemplary embodiment, a droplet test was carried out, according to which the coefficient of permeability for water lies at 5*10⁻⁷ m/s. For another exemplary embodiment, a droplet test was carried out, according to which the coefficient of permeability for water lies at 2*10⁻⁵ m/s.

The invention also discloses a method for propagating or culturing of biological material, comprising the introduction of fluid media, in particular the introduction of biological material or of a precursor of biological material, into a bioreactor, such as described above, and the recording of a physical, chemical, or biological measured variable with use of a holder for the sterile holding of at least one sensor, such as described above.

The invention also discloses a method for propagating or culturing biological material, comprising the introduction of fluid media, which contain biological material, or a precursor of biological material, into a bioreactor, in particular into a container of the bioreactor for receiving fluid media that contain biological material, wherein the container for receiving fluid media that contain biological material has a passage with a passage opening between the interior of the container for receiving fluid media that contain biological material and the exterior of the container for receiving fluid media that contain biological material, and a holder for the sterile holding of at least one sensor at or in the passage of the container for receiving fluid media that contain biological material is put in place prior to the introduction of the fluid media that contain biological material or a precursor of biological material.

These methods can also comprise the production of pharmaceuticals, in particular of biopharmaceuticals.

When the bioreactor with its container for receiving fluid media that contain biological material and the holder for the sterile holding of at least one sensor are sterilized, while the holder for the sterile holding of at least one sensor is held at least partially in the passage opening of the passage, it is possible to prevent reliably any contaminations from entering after the sterilization.

This is also the case, in particular, when the bioreactor with its container for receiving fluid media that contain biological material and the holder for the sterile holding of at least one sensor are autoclaved, while the holder for the sterile holding of at least one sensor is held at least partially in the passage opening of the passage.

In order to prevent any damage to the thermally sensitive sensors, the bioreactor can be equipped with a sensor only after the holder for the sterile holding of at least one sensor is put in place and, in particular, can be equipped with a sensor only after the sterilization and/or the autoclaving.

Advantageously, the recording of sensor-based measured values of a sensor, which is arranged in a herein disclosed holder for the sterile holding of at least one sensor, can then occur.

When the piston of the holder for the sterile holding of at least one sensor is arranged in a first position during the sterilization or autoclaving and in a second position during the recording of sensor-based measured values, it is thereby possible to minimize the thermal load placed on a sensor that is arranged at the piston.

The herein disclosed method can also be used with great advantage for phototrophic or mixotrophic microorganisms that have been modified by mutagenesis and, in particular, can also be used for microalgae, yeast, and bacteria.

The invention will be explained in detail below on the basis of preferred exemplary embodiments and with reference to the appended drawings. The features named above and shown in the figures can each be realized individually or in any desired combinations for the bioreactor according to the invention, the holder according to the invention, or the method according to the invention.

### BRIEF DESCRIPTION OF THE DRAWINGS

FIG. 1 a sectional view of multi-use bioreactor and a holder taken along line A-A in FIG. 4;

FIG. 2 a view from the top of the holder of FIG. 1;

FIG. 3 a view of the holder of FIG. 1 taken along line B-B in FIG. 2;

FIG. 4 a view of the holder of FIG. 1 taken along line C-C in FIG. 2;

FIG. 5a a sectional view of another multi-use bioreactor and holder taken along line A-A in FIG. 8;

FIG. 5b is a magnified view of the holder FIG. 5a;

FIG. 6 a view from the top of the holder of FIG. 5a;

FIG. 7 a view of the holder of FIG. 5a taken along line B-B in FIG. 6;

FIG. 8 a view of the holder of FIG. 5a taken along line C-C in FIG. 6;

FIG. 9 a sectional view of another multi-use bioreactor and holder taken along line A-A in FIG. 12;

FIG. 10 a view from the top of the holder of FIG. 9;

FIG. 11 a view of the holder of FIG. 9 taken along line B-B in FIG. 10;

FIG. 12 a view of the holder of FIG. 9 taken along line C-C in FIG. 10;

FIG. 13 a sectional view of a third embodiment of a multi-use bioreactor and holder;

FIG. 14 a magnified view of the holder FIG. 13 taken at rectangle K of FIG. 13;

FIG. 15 a magnified view of another embodiment of the holder of FIG. 14; and

FIG. 16 a side view of a multi-use bioreactor with a holder of the present disclosure showing a container partially disassembled; and

### DETAILED DESCRIPTION

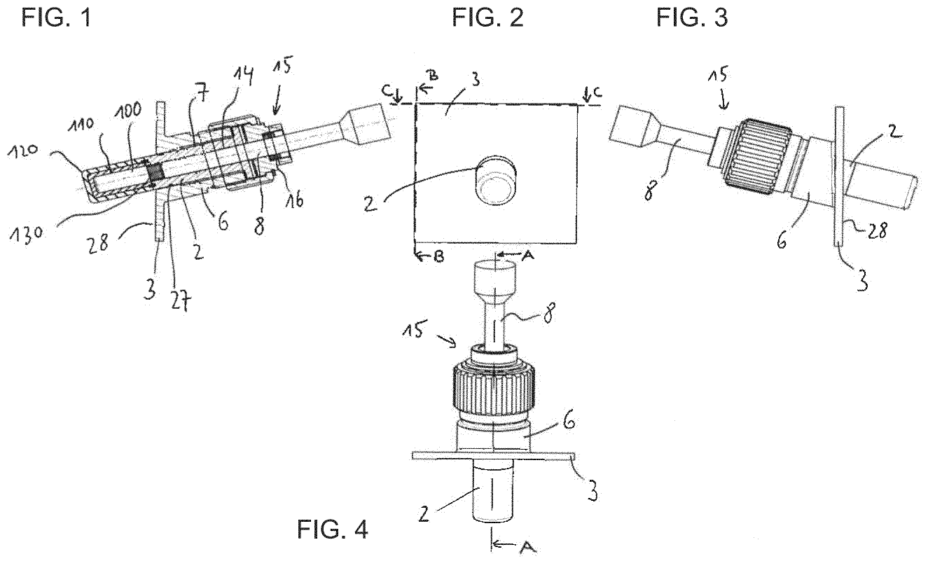

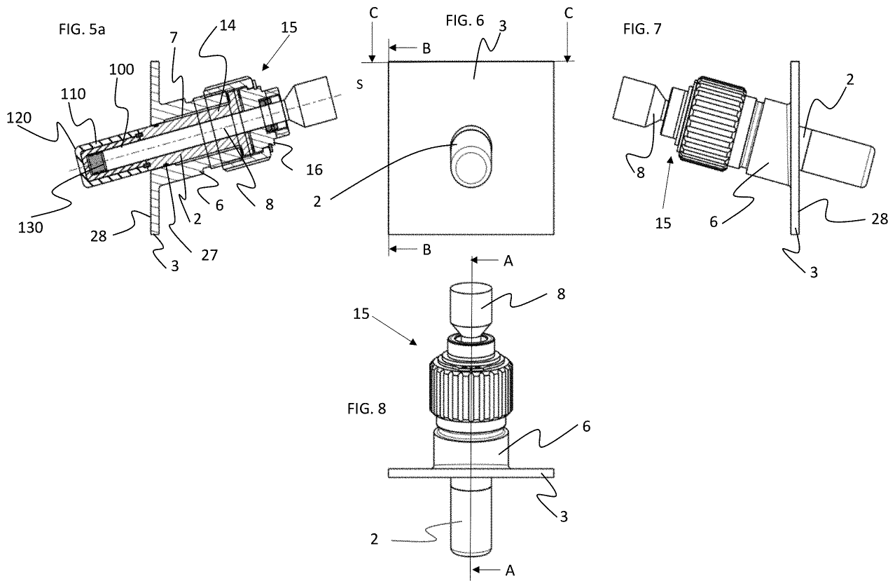

FIG. 1 a cross-sectional view of a first preferred embodiment of a multi-use bioreactor 1 and a holder 2 for the sterile holding of at least one sensor 8, which is arranged in a passage opening 7 of a container 3 for receiving fluid media 4 containing biological material 5, but which is illustrated only in part, wherein the sectional plane extends along the sectional plane AA illustrated in FIG. 4, in which a piston 130 of the holder 2 is shown in a position that is associated with a sensor-based recording.

FIG. 2 a view from the top of the first preferred embodiment of the holder 2 for the sterile holding of at least one sensor 8, which is arranged in a passage opening 7 of a container 3 that is intended for receiving fluid media 4 containing biological material 5, but is illustrated only in part, as viewed obliquely from above, but from the inner side or wall 28 of the container 3 for receiving fluid media 4, in which a piston 130 of the holder 2 for the sterile holding of at least one sensor is shown in a position that is associated with a sensor-based recording.

FIG. 3 a cross-sectional view, which illustrates the first preferred embodiment of the holder 2 for the sterile holding of at least one sensor 8, which is arranged in a passage opening 7 of a container 3 for receiving fluid media 4 containing biological material 5, but which is illustrated only in part, wherein the sectional plane extends along the sectional plane BB illustrated in FIG. 2, in which a piston 130 of the holder 2 for the sterile holding of at least one sensor 8 is shown in a position that is associated with a sensor-based recording.

FIG. 4 a cross-sectional view of the first preferred embodiment of the holder 2 for the sterile holding of at least one sensor 8, which is arranged in a passage opening 7 of a container 3 that is intended for receiving fluid media 4 containing biological material 5, but is illustrated only in part, wherein the sectional plane extends along the sectional plane CC illustrated in FIG. 2, in which a piston 130 of the holder 2 for the sterile holding of at least one sensor 8 is shown in a position that is associated with a sensor-based recording.

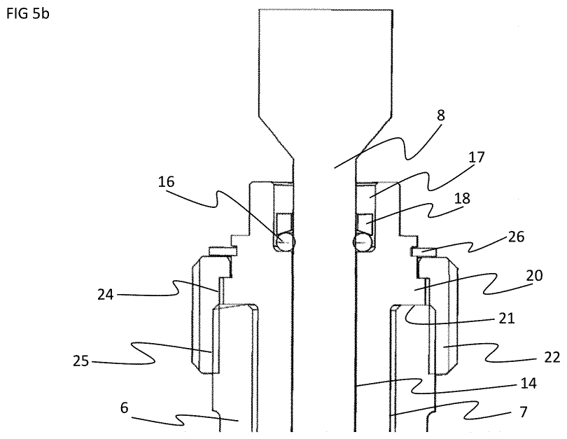

FIG. 5a a cross-sectional view of the first preferred embodiment of a multi-use bioreactor 1 and a holder 2 for the sterile holding of at least one sensor 8, which is arranged in a passage opening 7 of a container 3 that is intended for receiving fluid media 4 containing biological material 5, but is illustrated only in part, wherein the sectional plane extends along the sectional plane AA illustrated in FIG. 4, in which a piston 130 of the holder 2 for the sterile holding of at least one sensor 8 is shown in a position that is associated with a sterilization and/or an autoclaving.

FIG. 6 a view from the top of the first preferred embodiment of the holder 2 for the sterile holding of at least one sensor 8, which is arranged in a passage opening 7 of a container 3 that is intended for receiving fluid media 4 containing biological material 5, but is illustrated only in part, as viewed obliquely from above, but from the inner side 28 of the container 3 for receiving fluid media 4, in which a piston 130 of the holder 2 for the sterile holding of at least one sensor 8 is shown in a position that is associated with a sterilization and/or an autoclaving.

FIG. 7 a cross-sectional view of the first preferred embodiment of the holder 2 for the sterile holding of at least one sensor 8, which is arranged in a passage opening 7 of a container 3 that is intended for receiving fluid media 4 containing biological material 5, but is illustrated only in part, wherein the sectional plane extends along the sectional plane BB illustrated in FIG. 2, in which a piston 130 of the holder 2 for the sterile holding of at least one sensor 8 is shown in a position that is associated with a sterilization and/or an autoclaving.

FIG. 8 a cross-sectional view of the first preferred embodiment of the holder 2 for the sterile holding of at least one sensor 8, which is arranged in a passage opening 7 of a container 3 that is intended for receiving fluid media 4 containing biological material 5, but is illustrated only in part, wherein the sectional plane extends along the sectional plane CC illustrated in FIG. 2, in which a piston 130 of the holder 2 for the sterile holding of at least one sensor 8 is shown in a position that is associated with a sterilization and/or an autoclaving.

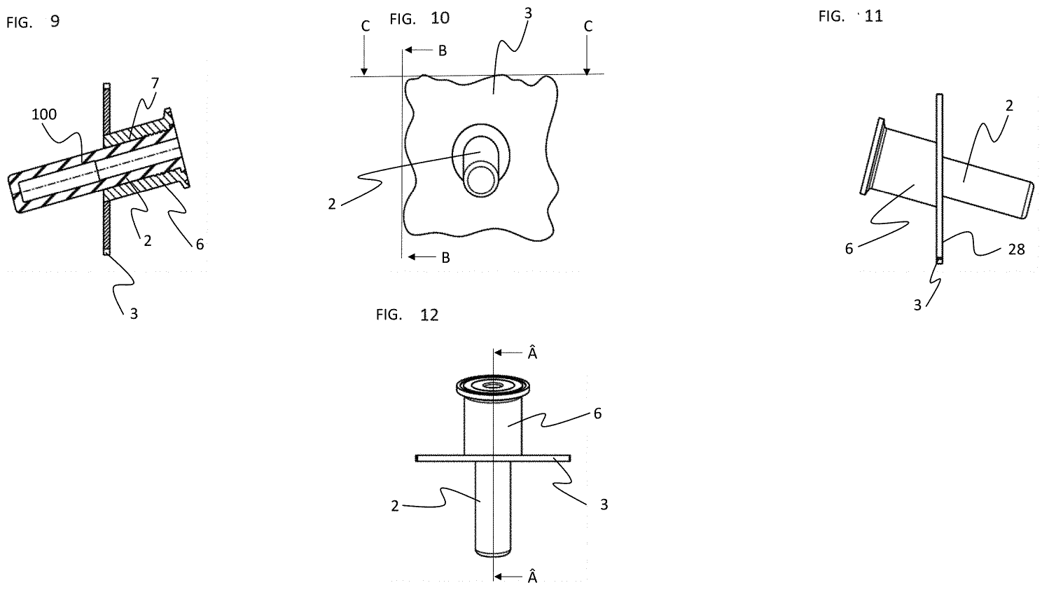

FIG. 9 a cross-sectional view of a second preferred embodiment of a single-use bioreactor 1 and a holder 2 for the sterile holding of at least one sensor 8, which is arranged in a passage opening 7 of a container 3 that is intended for receiving fluid media 4 containing biological material 5, but is illustrated only in part, wherein the sectional plane extends essentially as in FIG. 1 for the first embodiment, but without the piston 130 arranged in the sensor chamber 100 and without its piston rod.

FIG. 10 a view from the top of the second preferred embodiment of the holder 2 for the sterile holding of at least one sensor 8, which is arranged in a passage opening 7 of a container 3 that is intended for receiving fluid media 4 containing biological material 5, but is illustrated only in part, but as viewed from the inner side 28 of the container 3 for receiving fluid media 4.

FIG. 11 a cross-sectional view of the second preferred embodiment of the holder 2 for the sterile holding of at least one sensor 8, which is arranged in a passage opening 7 of a container 3 that is intended for receiving fluid media 4 containing biological material 5, but is illustrated only in part, wherein the sectional plane extends vertically through the container 3 for receiving biological material 5 in front of the holder 2 and in front of the passage 6, but without the piston 130 arranged in the sensor chamber 100 and without its piston rod.

FIG. 12 a cross-sectional view of the second preferred embodiment of the holder 2 for the sterile holding of at least one sensor 8, which is arranged in a passage opening 7 of a container 3 that is intended for receiving fluid media 4 containing biological material 5, but is illustrated only in part, wherein the sectional plane extends horizontally through the container 3 for receiving biological material 5 in front of the holder 3 and in front of the passage 6, but without the piston 130 arranged in the sensor chamber 100 and without its piston rod.

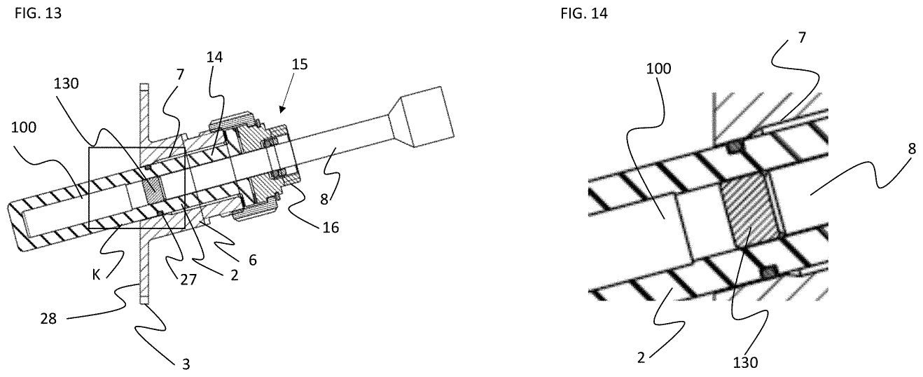

FIG. 13 a cross-sectional view of a third preferred embodiment of a multi-use bioreactor 1 and a holder 2 for the sterile holding of at least one sensor 8, which is arranged in a passage opening 7 of a container 3 for receiving fluid media 4 containing biological material 5, but which is illustrated only in part, wherein the sectional plane extends essentially as in FIG. 1 for the first embodiment.

FIG. 14 an excerpt of the rectangle K shown in FIG. 13 of a preferred embodiment in enlarged illustration, in which the piston 130 comprises an ethylene propylene diene monomer (EPDM) rubber.

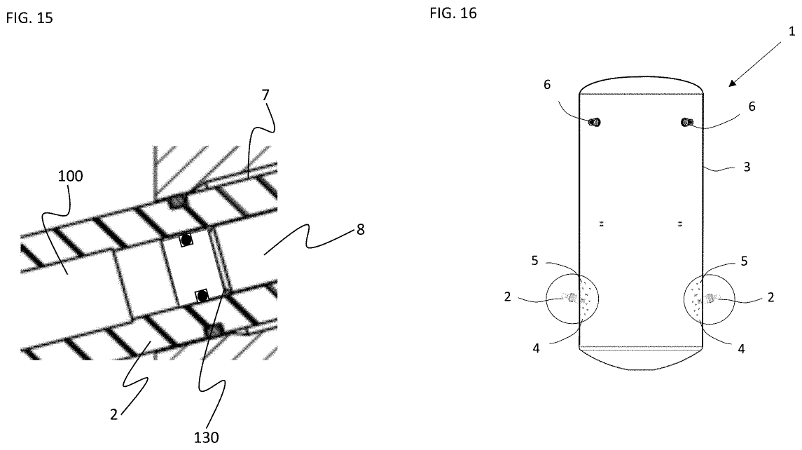

FIG. 15 an illustration, corresponding to the excerpt of the rectangle K shown in FIG. 13, of another preferred embodiment in enlarged illustration, in which the piston 130 comprises glass or is composed of glass.

FIG. 16 a side view of a multi-use bioreactor 1 with a holder 2 for the sterile holding of at least one sensor 8, in which the container 3 for receiving fluid media 4 that contain biological material 5 is shown partially exploded.

FIG. 5b is an excerpt from the illustration of FIG. 5a, which encompasses its top right area and is rotated in such a way that the line of symmetry S extends perpendicular to the horizontal.

A bioreactor 1 comprising a container 3 for receiving fluid media 4 that contain biological material 5 is provided. The reactor has a passage 6 with a passage opening 7 between the interior 28 of the container 3 for receiving fluid media 4 that contain biological material 5 and the exterior of the container 3 for receiving fluid media 4 that contain biological material 5. The reactor 1 has a holder 2 for the sterile holding of at least one sensor 8 extends at least partially in the passage opening 6 of the passage 6. The holder 2 for the sterile holding of at least one sensor 8 comprises a sensor chamber 100 and a fine-pored glass layer 110 with a pore size of up to 0.2 μm, wherein the fine-pored glass layer 110 with a pore size of up to 0.2 μm at least partially bounds the sensor chamber 100, whereby, in particular, an exchange of fluids between the interior 28 of the container 3 for receiving fluid media 4 that contain biological material 5 and the interior of the sensor chamber 100 is made possible.

The bioreactor 1 is further characterized in that the container 3 comprises stainless steel or is composed of stainless steel or comprises a plastic, in particular a sterilizable plastic, or is composed of a plastic, in particular, a sterilizable plastic.

The bioreactor 1 is further characterized in that the holder 2 extends inside the passage 6 in a form-fitting manner to the passage 6.

The bioreactor 1 is further characterized in that the bioreactor 1, together with the holder 2 can be autoclaved, in particular, can be autoclaved while it is held at least partially in the passage opening 7 of the passage 6.

A holder 2 for the sterile holding of at least one sensor 8 is provided that includes a sensor chamber 100 and a fine-pored glass layer 110 with a pore size of up to 0.2 μm, which bounds the sensor chamber 100 at least partially, whereby, in particular, an exchange of fluids between the interior 28 of the sensor chamber 100 and the exterior of the sensor chamber is made possible.

The holder 2 has the fine-pored glass layer 110 with a pore size of up to 0.2 μm surrounded by a second porous layer 120, which has a pore size of 200-700 μm with a porosity of up to 85%.

The holder 2 has the sensor chamber 100 with an essentially cylindrical shape and the first layer 110 and the second layer 120 extend in the longitudinal direction of the cylindrical sensor chamber 100 and, in particular, they extend distally to terminate at the front side 28.

The holder 2 has the first layer 110 is composed of a first glass or comprises a first glass and in which the second layer 120 is composed of a second glass or comprises a second glass.

The holder 2 has the first layer 110 is composed of a first glass or comprises a first glass and in which also the second layer 120 is composed of the first glass or comprises the first glass.

The holder 2 has the first layer 110 and the second layer 120 extend along the sensor chamber 100 in the longitudinal direction.

The holder 2 has the first layer 110 and the second layer 120 that do not extend continuously in the longitudinal direction, defining the sensor chamber 100, and are held on an essentially tubular support, in particular, are held on an essentially tubular support comprising a glass or composed of glass.

The holder 2 has a piston 130 that is displaceable in the longitudinal direction, in particular with a piston carrier or a piston rod, by means of which the piston is displaceable in the longitudinal direction.

The holder 2 has the piston 130 with a sensor holder. The holder 2 in which the piston 130 with sensor holder is displaceable between a first position, which is associated with a sterilization or an autoclaving, and a second position, which is associated with the sensor recording.

The holder 2 has the piston 130 has a place holder for at least one sensor 8.

The holder 2 has the piston 130 with a fastening—that includes mount 15, friction element 16, preferably O-ring, cylindrical recess 17, ring-shaped compression element 18, lateral shoulder 20 that extends in the radial direction, top flange 21, cap nut 22, thread 24, counter thread 25, snap ring 26, and sealing element 27, in particular O-Ring, for the sensor.

The holder 2 has the piston 130 that includes a plastic and, in particular, comprises an ethylene propylene diene monomer (EPDM) rubber or a ground glass piston, preferably with an O-ring arranged thereon.

LIST OF REFERENCE NUMBERS

| | |
|---|---|
| 1 | bioreactor |
| 2 | holder |
| 3 | container |
| 4 | fluid medium/media |
| 5 | biological material |
| 6 | port or passage |
| 7 | passage opening |
| 8 | sensor |
| 14 | passage opening |
| 15 | mount |
| 16 | friction element |
| 17 | cylindrical recess |
| 18 | compression element |
| 20 | lateral shoulder |
| 21 | top flange |
| 22 | cap nut |
| 24 | thread |
| 25 | counter thread |
| 26 | snap ring |
| 27 | sealing element |
| 28 | inner wall or side |
| 100 | sensor chamber |
| 110 | first porous layer |
| 120 | second porous layer |
| 130 | piston |

What is claimed is:

1. A bioreactor for fluid media that contain biological material, comprising:
a container having an interior configured to hold the fluid media and an exterior;
a passage with a passage opening between the interior and the exterior; and
a holder that extends at least partially in the passage opening, wherein the holder comprises a sensor chamber and a fine-pored glass layer with a pore size of up to 0.2 μm, wherein the fine-pored glass layer at least partially bounds the sensor chamber to allow an exchange of fluid between the interior of the container and the sensor chamber.

2. The bioreactor of claim 1, further comprising a sensor in the sensor chamber.

3. The bioreactor of claim 1, wherein the container comprises a material selected from a group consisting of stainless steel, plastic, and sterilizable plastic.

4. The bioreactor of claim 1, wherein the holder extends inside the passage in a form-fitting manner.

5. The bioreactor of claim 1, wherein the container, passage, and holder are autoclavable while the holder is held at least partially in the passage opening.

6. A holder for the sterile holding of sensor, comprising:
a sensor chamber having an interior and an exterior; and
a fine-pored glass layer with a pore size of up to 0.2 μm, the fine-pored glass layer bounding the sensor chamber at least partially to allow an exchange of fluids between the interior and the exterior.

7. The holder of claim 6, further comprising a second porous layer surrounding the fine-pored glass layer, wherein the second porous layer has a pore size of 200-700 μm with a porosity of up to 85%.

8. The holder of claim 7, wherein the sensor chamber is a cylindrical sensor chamber and the fine-pored glass layer and the second porous layer extend in a longitudinal direction of the cylindrical sensor chamber.

9. The holder of claim 7, wherein the fine-pored glass layer comprises a first glass and the second porous layer comprises a second glass.

10. The holder of claim 9, wherein the first glass and second glass are the same or different.

11. The holder of claim 7, wherein the first layer and/or the second layer do not extend continuously in the longitudinal direction.

12. The holder of claim 11, wherein the first layer and/or the second layer are held on a tubular glass support.

13. The holder of claim 8, further comprising a piston in the cylindrical sensor chamber, the piston being displaceable in the longitudinal direction.

14. The holder of claim 13, wherein the piston further comprises a sensor holder.

15. The holder of claim 13, wherein the piston is displaceable between a first position associated with sterilization or autoclaving and a second position associated with the sensor recording.

16. The holder of claim 13, wherein the piston comprises a material selected from a group consisting of plastic, ethylene propylene diene monomer (EPDM) rubber, and ground glass.

17. A method for propagating or culturing biological material in a fluid media, comprising:
providing a bioreactor having an interior configured to hold the fluid media and an exterior, the bioreactor having passage with a passage opening between the interior and the exterior and a holder that extends at least partially in the passage opening, the holder having a sensor chamber with a sensor and a fine-pored glass layer that partially bounds the sensor chamber to allow an exchange of fluid between the interior of the container and the sensor;
introducing the fluid media having a biological material or a precursor of a biological material into the bioreactor; and
using the sensor to record a physical, chemical, or biological variable of the fluid media and/or the biological material.

18. The method of claim 17, further comprising, prior to introducing the fluid media into the bioreactor, sterilizing the interior of the bioreactor, the sensor chamber, fine-pored glass layer, and sensor.

19. The method of claim 18, wherein the step of sterilizing comprises autoclaving.

20. The method of claim 18, further comprising moving the sensor in the sensor chamber from a first position during the sterilizing and a second position during the using the sensor to record.
